(19) United States
(12) Patent Application Publication
(10) Pub. No.: US 2004/0186356 A1
(43) Pub. Date: Sep. 23, 2004

(54) Surgical retractor and tissue stabilization device

(76) Inventors: Michael T. O'Malley, Ontario (CA); Michael S.G. Bell, Ontario (CA); Timothy Maxwell, Ontario (CA); James Henderson, Ontario (CA)

Correspondence Address: JOHN S. PRATT, ESQ, KILPATRICK STOCKTON, LLP, 1100 PEACHTREE STREET, ATLANTA, GA, 30309, US

(21) Appl. No.: 10/484,946

(22) PCT Filed: Aug. 5, 2002
(86) PCT No.: PCT/IB02/03037

Related U.S. Application Data
(60) Provisional application No. 60/310,829, filed on Aug. 9, 2001.

(51) Int. Cl. A61B 1/32

(52) U.S. Cl. 600/231

(57) ABSTRACT

Systems and methods for manipulation and stabilization of tissue and other structures during surgical and other specialized procedures. Components of the systems of this invention are widely applicable in both veterinary and human medicine, including procedures involving small or delicate structures or tissues requiring precise manipulation or stabilization, procedures requiring substantial retractive forces without causing tissue damage and procedures that benefit from exoskeletal retraction. Fixation components, which couple dynamic components and retraction or positioning components to base components, are easily adjustable, allowing manipulation of the system with one hand. Length and orientation of retraction or positioning components is also easily adjustable in several ways. Systems provide force appropriate for use in surgical procedures affecting delicate or small tissues or structures and provides a traumatic engagement, so that damage to tissues or structures is minimized, while adequate force is supplied and fine manipulation of the components is permitted.

### RELATED APPLICATION DATA

This application claims the benefit of U.S. Provisional Application No. 60/310,892 filed Aug. 8, 2001.

### FIELD OF THE INVENTION

This invention relates to systems and methods for manipulation and stabilization of tissue and other structures during surgical procedures, including procedures involving delicate or small structures, tissues requiring precise manipulation and stabilization, and other specialized procedures.

### BACKGROUND

In general, surgery and surgical treatment involve one or both of tissue separation and tissue joining. In surgery, medical treatment, and medical research, it is desirable to retract tissue, stabilize tissue, and present tissue in a variety of specific orientations to provide access to the area under investigation or repair, ideally in a method that creates minimal trauma beyond what is necessary for exposure and visualization of the operative area. In other words, it is desirable to exert a force on a tissue structure by reference either to some or all of the other tissue of which it is to become a part, as in the case of a transplant. Such an exertion of force for the purpose of tissue manipulation may be accomplished through very simple and short series of elements or through complex and lengthy series of elements that may or may not include gravity as a significant element. Examples of simple series in which gravity is not a significant element include sutures and staples (tissue joining) and a rib spreader (tissue separation).

In surgery and medical treatment it is frequently desirable to manipulate or stabilize tissue or other anatomical structures by, for instance, retracting tissue, approximating vessel or nerve ends, or reapproximating retracted or displaced tissue. During typical surgical procedures, tissue is usually incised, dissected, and retracted to gain exposure to the operative site, which, in some cases, may include structures deep in tissue requiring complex excavation through both hard and soft tissues to gain the required access. The principle ways this has been done historically include use of surgical assistant acting on a retractor or a retractor mounted on a fixed support assembly. Traditional retraction systems typically utilize mechanical fasteners to provide a rigid connection among components. Re-positioning the retractor may require additional equipment to change retractive or stabilizing forces. Additional tools may also be required to assemble or to disassemble equipment. This presents problems because it is difficult to add or change equipment in an operating room without compromising the sterile environment. As a result, surgical procedures can be delayed while additional sterile equipment is introduced to the operating facility.

A variety of needs also arise in connection with surgery to fix the position of tissue or structures, some of which “resist” repositioning or maintenance of a selected position. These needs are conventionally addressed with adhesive, such as by use of adhesive tape, and by use of devices that mechanically connect or attach, such as clamps and retractors. Another problematic shortcoming of traditional fixation systems is their reliance on threaded or incremental adjusters. Threaded adjusters are frequently too slow for mid-procedure adjustment. Incremental adjusters are faster but often exert too little or too much retraction between each pre-set detent position.

Advances in surgical techniques have created the need for a surgical system that can be manipulated by the surgeon in the course of the procedure. This is often necessary to provide clear and varied views of the incision during the procedure. Traditional systems and practices require the presence of an assistant for the duration of the procedure to provide and adjust retraction. As a result, procedure errors can occur because of misunderstood verbal communication between the surgeon and the assistant. Thus, it is desirable for the surgeon to be able to manipulate the apparatus and is preferable that it be possible to do so with one hand.

Although elements of many new technologies have been transferred to medicine from their original fields, this has generally not happened in the area of basic surgical instruments, even though surgery still largely depends on the skill of an individual surgeon using these tools. Recent research and development activity in medical equipment has been more focused on expensive procedure sets, diagnostic tools, and life-support systems. As a result, conventional surgical systems have changed slowly and suffer from a number of shortcomings.

One recent system for retraction and fixation of tissue, the CHESS™ system, portions of which are described in U.S. application Ser. No. 09/857,246, which is incorporated herein by reference, utilizes ferromagnetic components, fixation components, retraction components, and dynamic components in various combinations to provide a fixation and retraction system that provides stability while allowing efficient, sterile, relatively effortless adjustment of the system prior to or during a surgical procedure. While this prior system is effective for certain procedures, there are situations where a differently sized system adapted to exert different or delicate forces is needed and situations where specialized components are required.

### SUMMARY OF THE INVENTION

Components of the system of this invention are widely applicable in both veterinary and human medicine, including procedures involving small or delicate structures or tissues requiring precise manipulation or stabilization, procedures requiring substantial retractive forces without causing tissue damage and procedures that benefit from exoskeletal retraction. Exoskeletal retraction is retraction using the patient or related structure, such as a bed or operating table, as an anchor or base. Generally, systems of this invention provide one or more of stabilization, manipulation, retraction and approximation. These general actions may include many sub-categories of function, including presentation of tissue in specific orientation. Fixation components, which couple dynamic components and retraction or positioning components to base components, are easily adjustable, allowing manipulation of the system with one hand. Length and orientation of retraction or positioning components is also easily adjustable in several ways. This system provides a force appropriate for use in surgical procedures affecting delicate or small tissues or structures and provides a traumatic engagement, so that damage to tissues or structures is minimized, while adequate force is supplied and fine manipulation of the system is permitted.

This invention provides many advancements and advantages over current small animal surgical techniques, including the ability to quickly stabilize and retract small animal tissue for a surgical procedure while affording the surgeon precise control and easy manipulation of both the animal and the retracted tissue, and provides advantages for use in human surgical procedures requiring delicate force and manipulation. In one embodiment, this system is compatible with animal body temperature maintenance systems in both aspirated or intravenous anesthesia systems.

Other features of this invention include a microsurgical system, allowing the retrieval, stabilization, and holding of small vascular or neurological vessels and providing presentation, permitting the surgeon to reapproximate the severed ends.

Yet another feature of this invention is a system that provides anchor points attached to the body using an adhesive. Another feature is a traumatic retraction of tissue. Finally, this invention provides systems using exoskeletal retraction.

Systems of this invention both set up and disassemble quickly, are easily cleaned and are autoclavable. Finally, systems offer both static and dynamic retraction and provide a wide range of finely-formed retractors that are a traumatic to tissue or other structures.

These and other features of this invention will be readily understood by those skilled in the art by reference to the following descriptions of the invention and the accompanying drawings.

### BRIEF DESCRIPTION OF THE DRAWINGS

FIG. 1 is a perspective view of a microsurgical system according to one embodiment of this invention.

FIG. 2 is a perspective view of a small animal surgical system according to one embodiment of this invention.

FIG. 3 is a perspective view of a surgical system according to one embodiment of this invention.

FIG. 4 is a top view of a base component according to one embodiment of this invention.

FIG. 5 is a top view of a base component according to another embodiment of this invention.

FIG. 6 is a perspective view of the base component of FIG. 3.

FIG. 7 is a perspective view of a system according to an alternative embodiment of the invention.

FIG. 8 is a perspective view of the system of FIG. 7 in place on a patient.

FIG. 9 is a perspective view of a system according to an alternative embodiment of the invention.

FIG. 10 is a side view of the fixation component of FIGS. 1 and 2.

FIG. 11 is a side view in partial cross section of the fixation component of FIG. 10.

FIG. 12 is an exploded view of the fixation component of FIG. 10.

FIG. 13 is a perspective view of the fixation component of FIG. 10 together with a retraction component of this invention.

FIG. 14 is a perspective view of the fixation component of FIG. 10 together with a positioning component of this invention.

FIG. 15 is a perspective view of the fixation component of FIG. 10 together with a retraction component and a dynamic component of this invention.

FIG. 16 is a side view of an alternative embodiment of the fixation component of FIG. 10.

FIG. 17 is a perspective view of an alternative fixation component according to one embodiment of this invention and as shown in the system of FIG. 3.

FIG. 18 is a perspective view of the fixation component of FIG. 17 together with a retraction component of this invention.

FIG. 19 is a perspective view of the fixation component of FIG. 17 together with a retraction component a dynamic component of this invention.

FIGS. 20-25 are perspective views of alternative embodiments of retraction components of this invention.

FIG. 26 is a perspective view of a saddle retractor of this invention

FIG. 27 is a top view of the retractor of FIG. 26.

FIGS. 28 and 29 are perspective views of various aspects of the system of FIG. 1.

### DETAILED DESCRIPTION OF SPECIFIC EMBODIMENTS

Overview

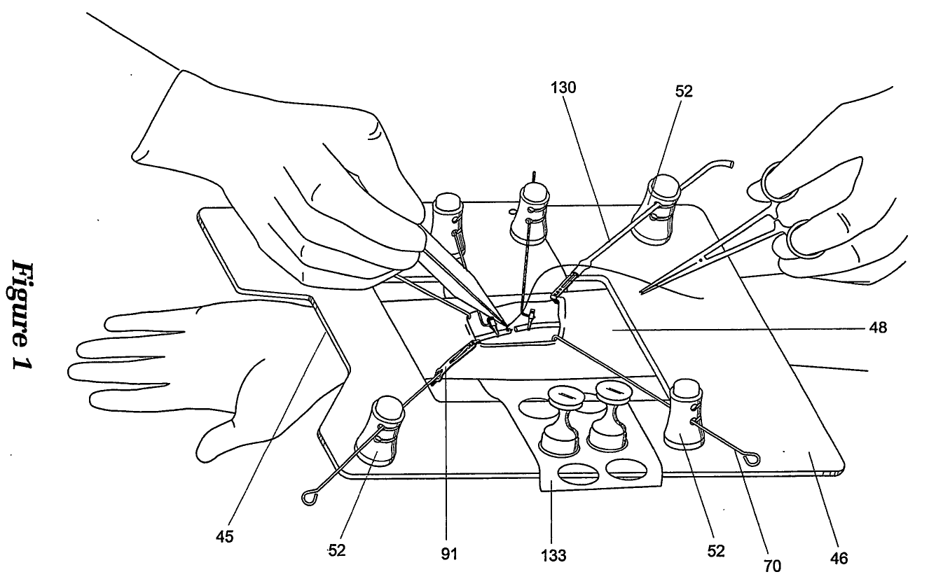

Stabilization and manipulation systems according to this invention may be utilized during a surgical procedure for manipulation, stabilization, fixation, immobilization, or retraction of structures such as, but not limited to, flesh, skin, bone, fur, hair, vessels, nerves and other human and animal tissues or structures. The system generally may include base components, fixation components, retraction and positioning components, and dynamic components. Various components may be used for coupling a fixation component to tissue or other structure. Combinations of the components form a variety of magnetically and mechanically coupled structures and systems, such as the systems illustrated in FIGS. 1-3 and further described below. Some systems are fully steam autoclavable up to at least 284° F.

Base Components

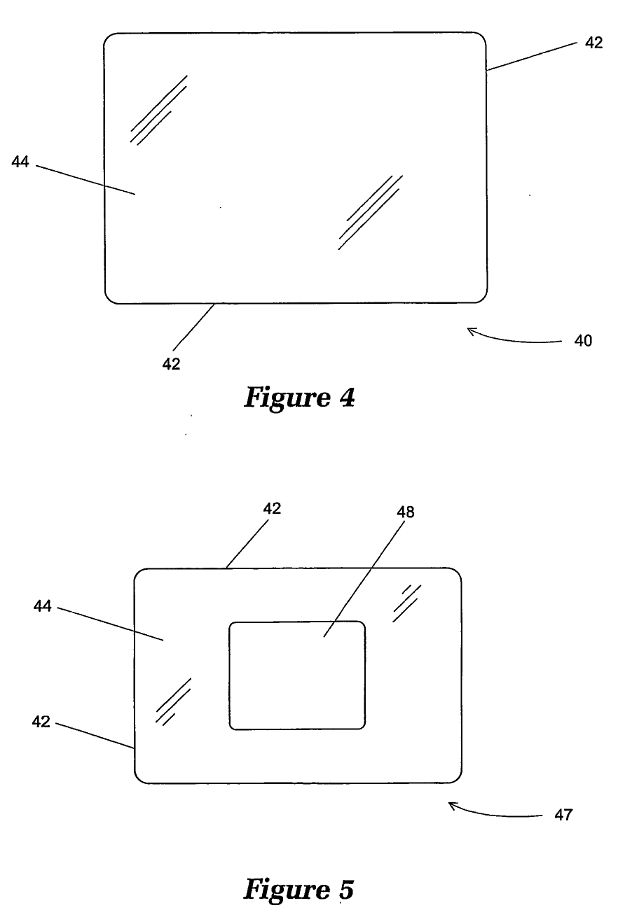

One example of a base component is a stainless-steel plate 40, shown in FIG. 4, which is essentially the surgical operating table or base. The plate 40 may be formed from 0.065 inch thick stainless steel. The plate has four edges 42 and a smooth, flat stainless steel surface 44. The base component may have a non-reflective surface finish. One or both faces of the plate 40 may be laser etched or otherwise permanently marked with text or diagrams (not shown) so that it is a complete instructional tablet. This is advantageous because traditional paper instructions tend to be lost, so that advanced functionality is forgotten and therefore effectively lost over time.

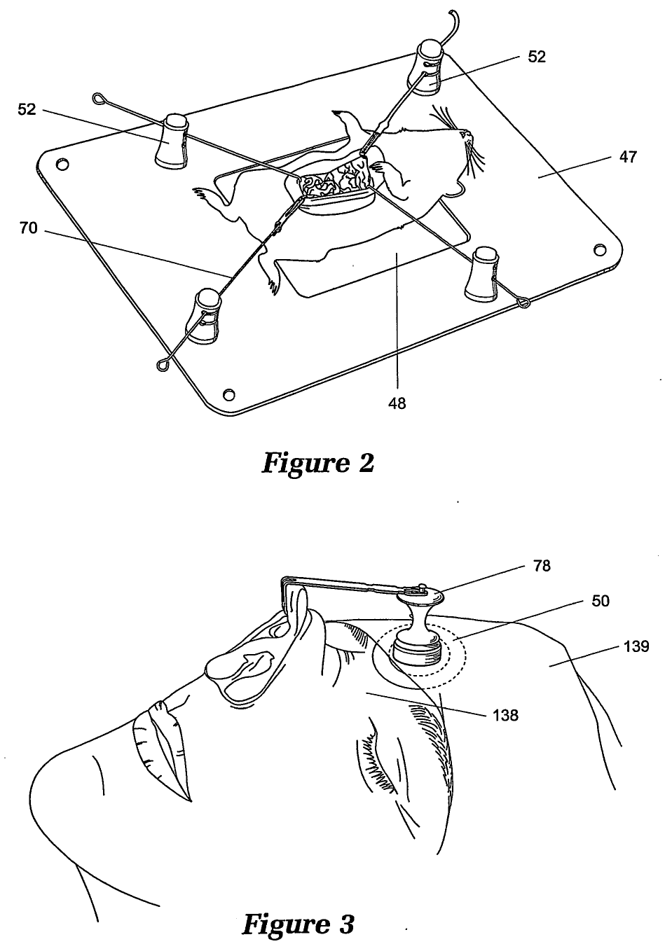

Other base plates, such as base plate 46, shown in FIG. 1, and base plate 47, shown in FIG. 5, include window 48. Base plate 46 may be placed over the surgical site, as shown in FIG. 1, so that the incision may be viewed and accessed through the window 48. Base plate 46 includes cutout 45. Alternatively, as shown in FIG. 2, a small animal may be placed on the window 48 of base plate 47, so that the animal may be placed directly on a warming surface. In this manner, the system may be compatible with animal body temperature maintenance systems in both aspirated or intravenous anesthesia systems and may provide a radiolucent area for imaging.

Various sizes of base plate 40, 46 or 47 may be provided in order to tailor the system for a particular surgical procedure. For example, a base plate 46 for use (with, for example, a mouse) in a small animal surgical system may be 12 inches by 8 inches, and the window 48 may be 5 inches by 4 inches. In another embodiment for use with a slightly larger animal, such as a rat, the plate may be 14 inches by 10 inches and the window may be 10 inches by 4 inches. Base components for use in microsurgical applications may vary in size and shape depending on the use. One side of a base component may be slightly curved or include a cutout for placement on a wrist, as shown of plate 46 in FIG. 1.

Any base plate of this invention may be covered with microfiber textile surgical fabric, a self-adhesive barrier film, or used bare. Fabric aids in movement of the fixation components, described below, by decreasing the coefficient of friction. Thus, through their functional range, stationary to full motion, the fixation components provide near linear resistance. This permits the surgeon to make fine adjustments using two fingers to slide the fixation components along the operating surface with a linear and predictable force. At rest, a fixation component indents the fabric slightly so that the fabric is slightly depressed and does not detract from the fixation component's vertical attraction for the plate. The fabric may be made from a microfiber that has substantially reduced blood transmission characteristics over normal fibers and is washable a large number of times. The fabric may be obtained from Burlington Cloppman and is generically referred to as a microfiber fabric. An example of a suitable microfiber is one that is 99% polyester and 1% carbon fiber. The fabric may be attached to the plate by a crack and peel tape system or by battens and fixation components. In an alternative embodiment, the plate may be coated or treated to achieve the functional benefits of the described fabric. Alternatively, the fixation components may be applied to the base plate through a sterile surgical drape.

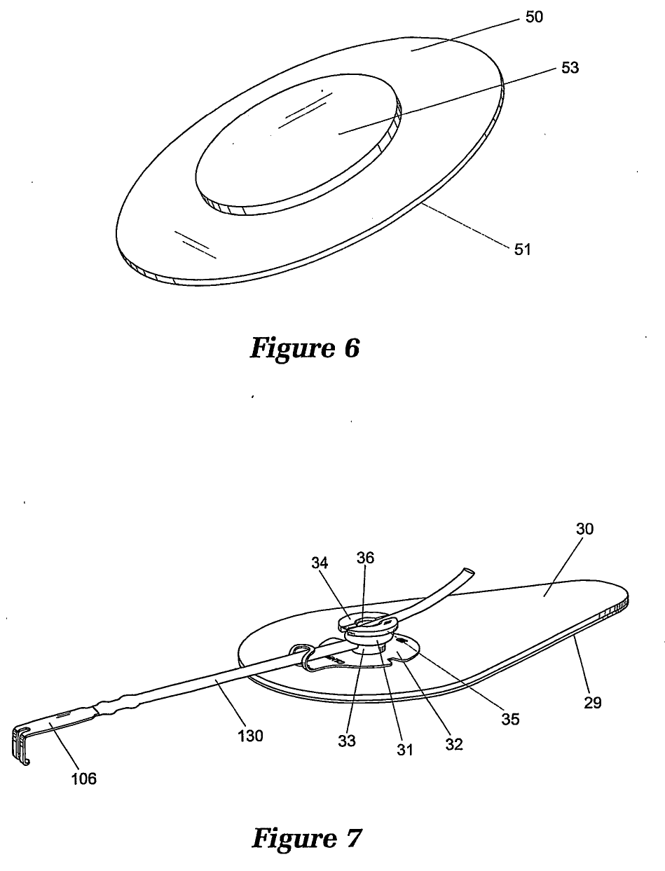

As shown in the system illustrated in FIG. 3, base components may include at least one plate 50 attached to the patient using a suitable adhesive. Plate 50, shown in detail in FIG. 6, includes adhesive layer 51 and coin 53. Coins 53 may be manufactured from ferromagnetic steel, or other suitable magnetic material. Adhesive plate base components 50 are generally smaller than the base components 40, 46 and 47 shown in FIGS. 1, 4 and 5, and are useful in tissue manipulation and stabilization of areas such as the head, face, neck, shoulders and sternum.

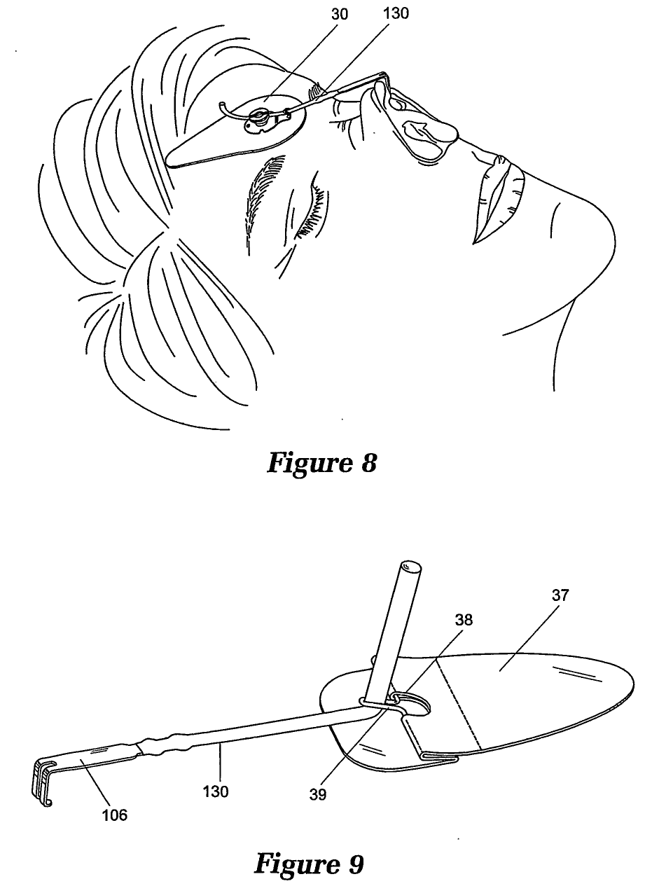

An alternative embodiment of an adhesive base component, shown in FIGS. 7 and 8, plate 30, includes adhesive layer 29, rivet 31 that extends from the body 32 and includes a post 33 and a cap 34. Cap 34 includes rim 35 and conical sections (not shown). Slot 36, which extends through cap 34 and partially into post 33 so that post 33 is split by slot 36, is adapted to receive a dynamic component, such as elastic tube 130, further described below, which may also be wrapped around at least part of post 33. The elastic tube thus contacts a substantial portion of the anchor surfaces by passing first through the slot in the post and then wrapping around a significant portion of the circumference of the post. Wrapping the elastic tube around, in effect, a 90° corner as the tube exits the slot causes the tube to flatten at the comer, establishing substantial surface contact between the tube and the rivet, thereby resisting slippage between the two. The elastic tube may also wrap around a second comer and pass through slot 36 in post 33 a second time, securing its position. The teardrop shape of this base component allows anchors to be placed close together on the inside of a curve. In an alternative embodiment, such as plate 50 in FIG. 3, the adhesive base is circular. Any other suitable shape may also be used.

In another alternative embodiment of an adhesive base component, shown in FIG. 9, a woven or non-woven textile tape 37 with an aggressive skin adhesive is folded to entrap a wire bar that protrudes through a hole 38 in the tape, forming a locking wire 39 that may secure a dynamic component, such as elastic tube 130.

In alternative embodiments, the base component may be made from non-magnetic material, such as copper, aluminum, some stainless steel and other alloys, most plastics, or other suitable material to which a magnet is not attracted. Alternatively, the base component may include magnetic plates or strips to which magnetic fixation components are attracted. These plates or strips may be located in the base component surface, or under the surface in such a manner to allow the fixation components to adhere.

In another embodiment, such as for use with for brain retraction or abdominal surgery, an articulated arm is attached to the operating room table or to a bedrail, or other suitable structure, providing a base component surface for fixation components. In this embodiment, it is possible to locate and secure a work surface anywhere in free space around the patient and attach the system components to the work surface. The work surface is attached to the table or bedrail using a movable arm that may be made from a flexible coiled steel goose neck strong, enough to provide fixation, but flexible enough to be manually positioned and manipulated. The arm may be designed having rigid sections that are interlinked with locking joints, or in any other suitable configuration.

Fixation Components

Fixation components of this invention may contain rare-earth magnets and may attach to a base component or to another ferromagnetic component. Alternatively, the fixation components may be weighted so that they rest on a base component, or some other suitable surface, and anchor to the base through mass, surface area, and friction. System fixation components work on both bare and draped plates. In alternative embodiments, fixation components are suspended or free hanging weights.

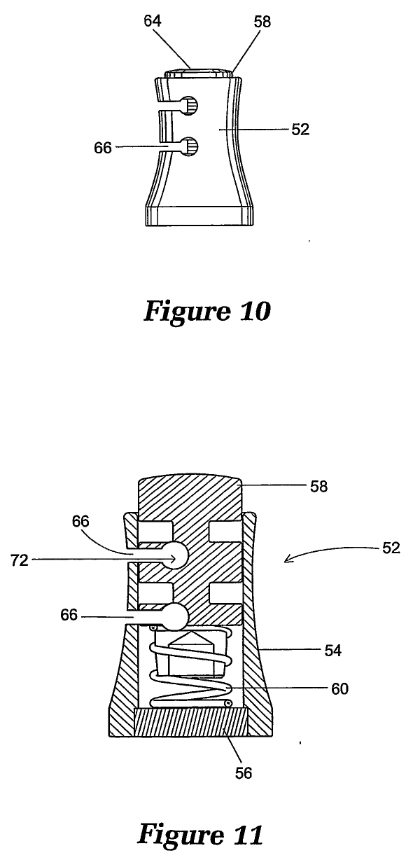

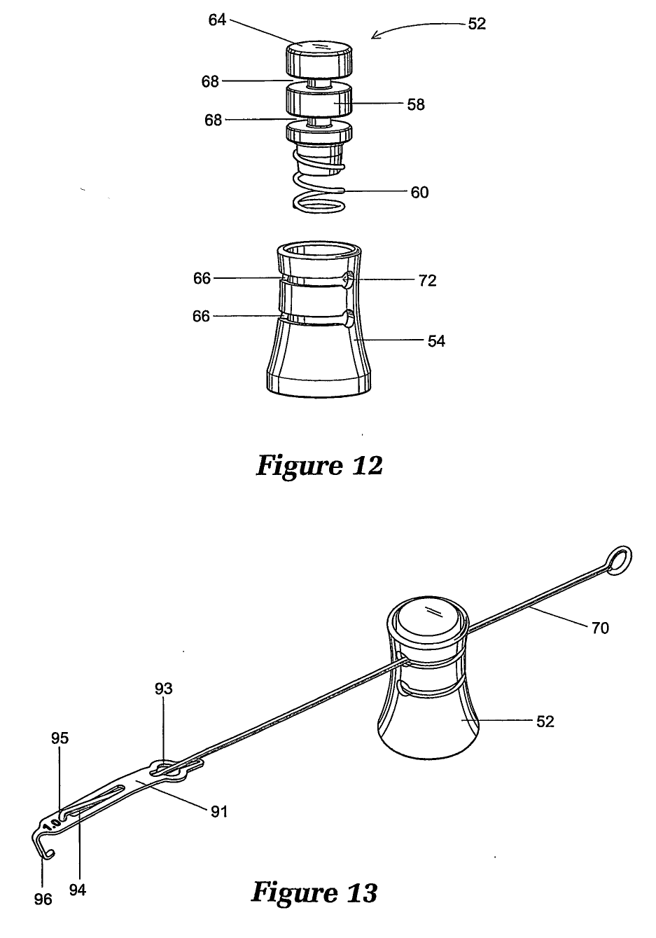

Magnetic fixation component 52, shown in FIGS. 1 and 2 and illustrated in detail in FIGS. 10-12, includes a housing 54, a rare earth magnet 56, a plunger 58 and a biasing element. The biasing element may be a compression spring wound from stainless steel spring wire, such as spring 60, an elastomer, or another magnet with an opposing pole to the base magnet. The rare earth magnet 56 (typically a disk or cylindrical section) is positioned in the housing 54, which may be made from 6061-T6 aluminum finished with a hard anodized finish, or other suitable material. The plunger 58 may also be made from 6061-T6 aluminum finished with a hard anodized finish, or other suitable material. In an alternative embodiment, the magnet is housed in cup, which is positioned in the housing, maximizing magnetic force downward and minimizing magnetic force radiated upward. The magnet may be neodymium iron boron, or other suitable rare earth magnet, and may be plated with a low nickel coating so that repeated autoclaving does not cause finish degradation.

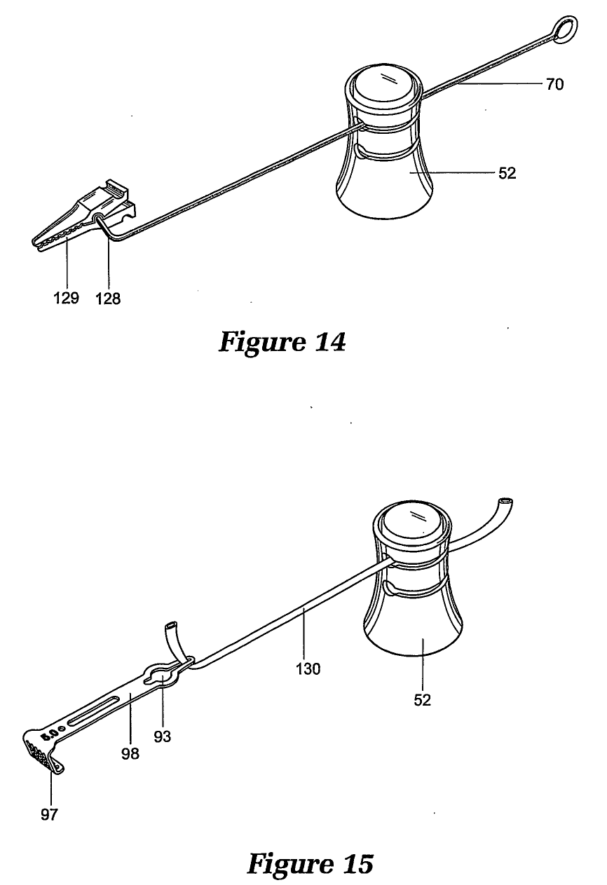

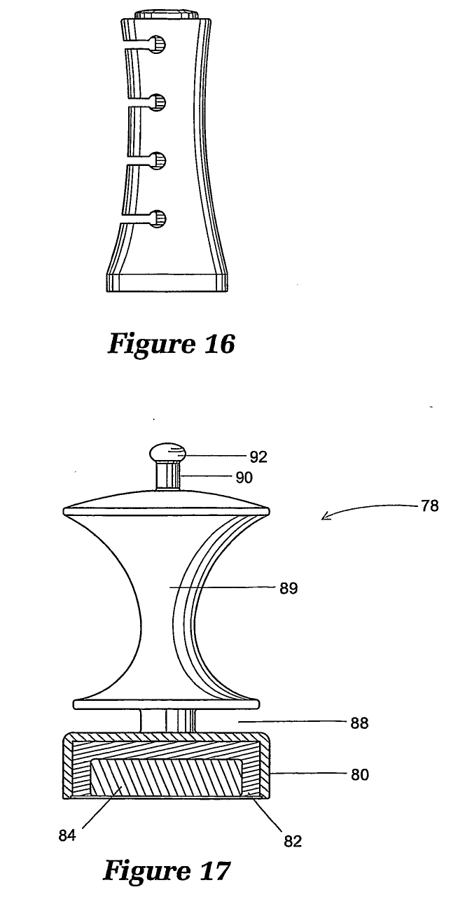

Fixation component 52 may be manipulated in order to capture or release a wire or wire-retractor combination, as shown in FIG. 13, or a positioning component, as shown in FIG. 14, or to capture or release an elastomer, as shown in FIG. 15. Fixation components may also capture and release structures such as probe leads, and monofilament or nylon cords, allowing other devices to be secured and manipulated as part of the system. As may be understood by reference to FIGS. 10-12, button 64 of the plunger 58 is depressed so that one of the slots 66 of housing 54 and one of the cleats 68 of the plunger 58 are aligned, allowing insertion of a wire (or elastomer) into the aligned slot 66 and cleat 68 through to a cutout 72 in the housing 54. Release of the button 64 allows the spring 60 to force the plunger 58 upward, capturing the wire 70 between surface 76 of the body of the plunger 58 and the cutout 72 of the housing 54. This provides stable and secure capture and retention of the wire 70, while subsequent depression of the button 64 allows easy release of the wire 70. The spring 60 is held in position by magnetic attraction between the spring 60 and magnet 56. The length of the captured wire 70 (or other captured element) is easily and quickly adjustable simply by depressing the button 64, sliding the wire 70 to its desired location, and releasing the button 64. Wire 70 may also be rotated 360° and fixed in any orientation. The differing height of the slots 66 and cleats 68 allows a component to be captured at one of two heights. Alternatively, as shown in FIG. 16, the housing may have four slots. In this embodiment, the height of the housing is increased to about two inches to accommodate the increased number of slots. Alternative embodiments may include any number of slots, as desired, and the height may be adjusted to accommodate the number of slots.

Fixation component 52 acts as a spring activated lock that provides a gripping force on a wire 70, elastomer, or other suitable object. The surgeon is able to manipulate the wire 70 by depressing the button 64, thereby disengaging the locking mechanism, allowing the surgeon to shorten, lengthen and/or rotate the wire 70 as required or desired. As further described below, a wire 70 may act as a handle for a retraction component or positioning component, or may act as a retractor or positioner itself. A coupled fixation and retraction (or positioning) component may be anchored anywhere on a base component or other suitable surface and is easily repositioned as required. One end of a retractor or positioner may engage tissue, such as skin, or may grasp tissue or other structures such as vessels or nerves, further described below. The fixation component may be positioned to allow continued retraction or positioning of tissue by a retraction or positioning component. The wire may be bent into any configuration, as desired by the surgeon and as further described below.

An alternative embodiment of a fixation component 78 has a magnetic circuit design utilizing the components shown in FIG. 17 consisting of external housing 80, ferromagnetic cup 82, and rare earth magnet 84. Rare earth magnet 84 (typically a disk or cylindrical section) is housed in ferromagnetic cup 82 (typically, for instance, 12L14 steel), in turn housed in external housing 80, which external housing is a stainless steel (e.g., non-magnetic 304 stainless steel). Housing magnet 84 within ferromagnetic cup 82 focuses the magnetic field so that the magnetic force downward is maximized, while the magnetic force radiated upward is minimized. The narrow waist of the component and the cleat, described below, also serve to maximize magnetic force downward and minimize magnetic force radiated upward.

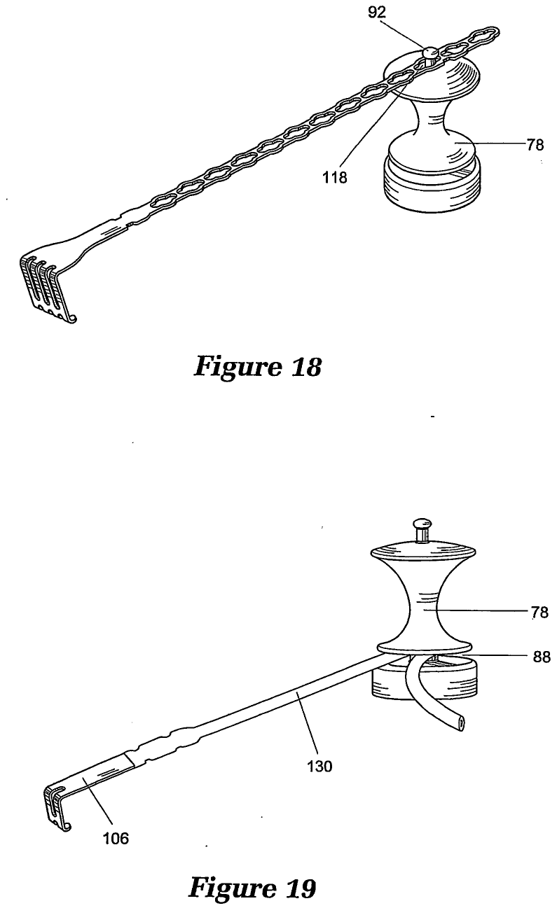

As shown in FIG. 17, a fixation component of this embodiment may have a cleat 88 in the form of a deep annular groove between the external housing 80 and a body 89. A boss 90 is positioned on the surface of body 89 and serves as an attachment point for a retraction or dynamic component. Boss 90 has elliptical nob top 92 that retains a retraction component, as shown in FIG. 18, or that captures a dynamic component. Cleat 88 can grip the edge of a base plate or be used to capture an elastic tube by wrapping the tube around the cleat, as shown in FIG. 19. Other fixation components according to this embodiment may have similar components and may vary in height according to system needs.

In some embodiments of this invention the housing and other structures are formed from non-magnetic material. Non-magnetic material includes material, such as copper, aluminum, some stainless steel and other alloys, and most plastics, to which a magnet is not attracted. In a plastic housing embodiment, the plastic may be molded to form a shell encasing the magnet or weight. All stainless steel components may be surgical stainless steel.

In one embodiment, a weighted fixation component acts as a ballasted anchor, using its mass and the coefficient of friction between the component and the surgical base to provide a stable fixation element. In this embodiment, the fixation component contains a housing, a non-magnetic weight, a plunger, and a biasing element. In this embodiment, the biasing element is held in place by a mechanism such as a notch or threaded neck.

An alterative embodiment of a fixation component of this invention includes a cam activated locking device on the fixation component. In this embodiment, the handle of the cam provides, through gravitational force, a locking mechanism that engages the retractor body, but allows adjustment in the traction direction while preventing movement in the opposite direction without activation of the cam by the surgeon. Other alternative embodiments include various mechanical entrapment mechanisms, such as a screw.

Retraction and Positioning Components

The retraction and positioning components of the system may be light, thin, economical instruments that perform their coupling function with minimal clutter while providing superb control and feel. The retraction and positioning components are typically 0.020 inch thick stainless steel, although other materials may also be used. This stainless steel is autoclavable at any temperature, or may be cleaned by any method approved for stainless steel instruments.

Generally, the retraction components have at one end a flexible hook or prong. As shown in FIG. 13, a retractor 91 may include a double keyhole opening 93 adapted to receive a wire 70, so that the wire passes through the double keyhole 93 and also through opening 94 and aperture 95, securing the wire 70 and the retractor 91 together. The wire 70 is captured by the fixation component 52 as described above. Double keyhole 93 may also capture a dynamic component, such as an elastic tube 130, as shown in FIG. 15. The retractor may also include a prong, such as single prong 96 of retractor 91, shown in FIG. 13, and wider prong 97 of retractor 98 shown in FIG. 15. In another embodiment, a prong includes a sharp tip, which may be used for skin piercing and wound edge reduction.

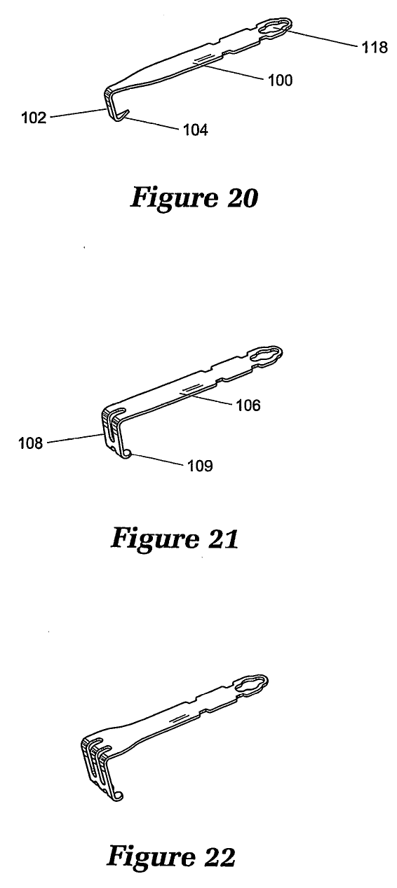

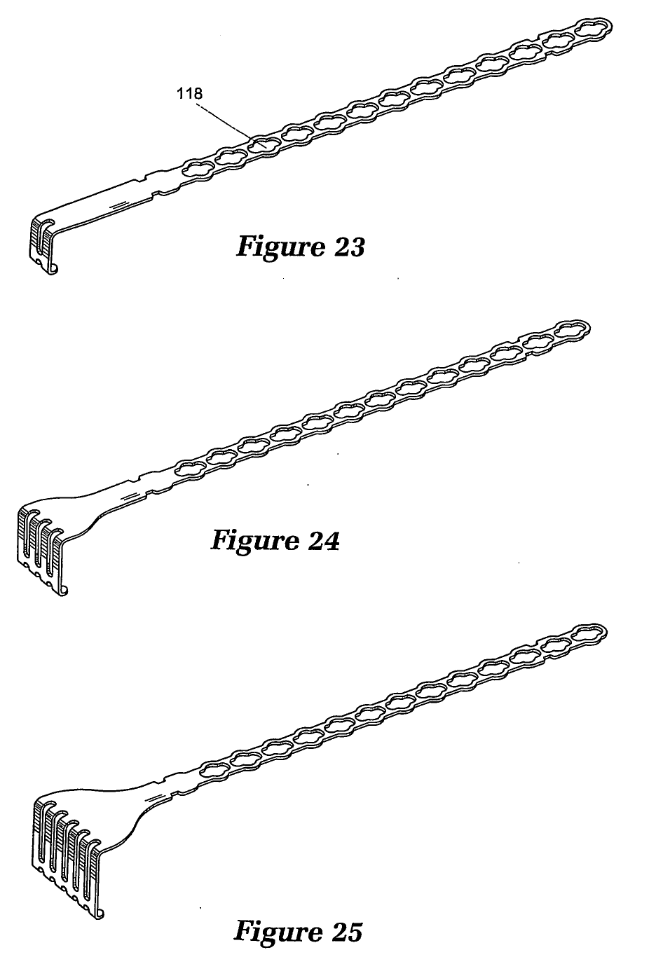

The embodiments shown in FIGS. 20-25 are generally for use with components such as fixation component 78, as illustrated in FIG. 18. As shown in FIG. 20, a retractor 100 may have a prong 102, including sharp tip 104. Other retractors, such as retractor 106 shown in FIG. 21, include a prong 108 having a blunt tip 109. The length of any prong of any of the retractors may be increased in proportion to the width of the prong to provide progressively deeper reach. The width of the prong may vary between about 1 mm to about 7.5 mm. The length of the retraction component may also vary, as illustrated in FIGS. 20-25.

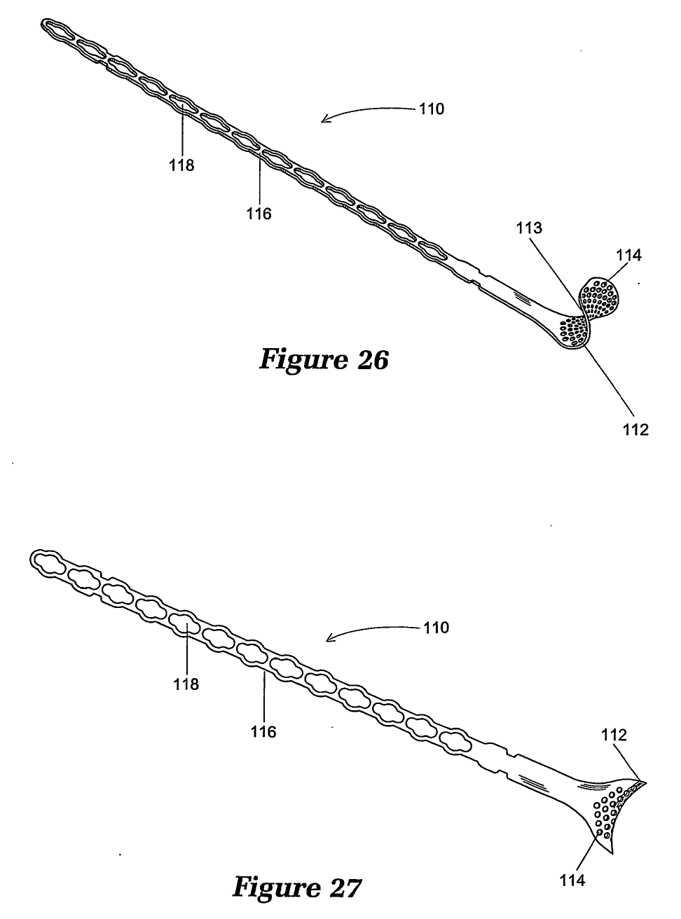

A saddle retractor 110, shown in FIGS. 26 and 27, provides a traumatic retraction of tissue, and includes curved end 112 having tissue contact surface 113, which surface 113 includes a plurality of apertures 114, which grip tissue by displacement. Saddle retractor 110 provides high retractive force without causing tissue trauma. The curved tissue contact surface 113 of saddle retractor 110, like a horse saddle, is curved in opposite directions along orthogonal axes. Perforating the tissue contact surface with holes or recesses such as apertures 114 provides a contact surface that is highly effective in engaging tissue. Curved surface 113 could also be described as a fragment of an inner portion of a toroid surface.

The retraction components may include an arm design of an interlock and key-holes. For example, as shown of saddle retractor 110 in FIG. 27, an elongated arm 116 is a strip of sheet metal perforated by a double-keyhole shaped cutouts 118 having at least two different widths. The “double key-hole” could also be described as an oblong opening centered on a round hole having a diameter larger than the oblong width but smaller than its length. Retractors may attach to a boss on a fixation component (FIG. 18), an elastic tube (FIGS. 15 and 19), a wire handle (FIG. 13), or another retractor (not illustrated).

The prong 102 or saddle curve 112 of a retraction component may hook an area of skin or tissue to be retracted. The retractor is then pulled into position, so that the wound is opened. The arm of the retractor is attached either to an elastic tube, to a handle, such as a wire or other conventional retractor handle, to another retraction component, or directly to a fixation component. The elastic tube or handle may attach to a fixation component and then to a base component so that the retractive position is secured. Adjustments may also be made simply by moving the fixation component on the base component.

Wire 70, shown in FIGS. 1, 2 and 14, includes ends 126, at least one of which may be bent to form a hook 128, such as an arcuate end or 80° angle end, in order to engage other components, such as a clamp 129, or other suitable device, thereby creating a microsurgical system, further described below. In this manner, the wire functions as a positioning component. Wire handles may also be formed into a variety of shapes either by the manufacturer or made with uniformed ends to be shaped by the user in order to create tissue engaging elements, such as hooks or loops, in the wire itself.

Using 0.020 inch sheet metal for the retractors and drawn stainless steel wire for the positioners provides flexibility and a sufficiently constant bend modulus to allow them to be cut or bent to form specialized instruments. For instance, the end of a retractor or wire may be bent so that it attaches directly to edge of a plate. In other embodiments, any of the retractors and positioners may be formed from plastic or any other suitable material. For instance, the saddle retractor 110 could be injection molded of suitable plastic.

Dynamic Components

The dynamic components of the system may be formed in cords, rods, tubes, bands, loops or other suitable structures. The components may be made from any suitable elastic material, including, without limitation, latex rubber, silicone, natural rubber and materials of similar elasticity, GR-S, neoprene, nitrile-butyl-polysulfide, ethylene-polyurethane, polyurethane, or any other suitable material. Generally, non-reactive materials such as silicone and hypoallergenic forms of latex are desirable.

A dynamic component, such as an elastic tube, may have a 0.125 inch diameter with a Poisson ratio and durometer that provide a secure mechanical lock onto a retractor. The elastic tube increases in diameter when compressed and decreases in diameter when stretched. This allows a portion of an elastic tube in a stretched state to be secured in a slot having a diameter smaller than the diameter of the elastic tube in a resting state, while the diameter of the portion of the elastic tube in a resting state remains greater than the slot diameter, thereby providing resistance. The elastic tube may be disposable and may have an 8:1 stretch modulus. These inherent qualities makes it easy to slide onto a retractor arm and yet lock in position under tension.

A dynamic component, such as elastic tube 130, may be threaded through the slit of a retractor, locking it in place, as shown in FIG. 15, or alternatively may slide over the end of a retractor, as shown in FIG. 19. Elastic tube 130, or other dynamic component, may be captured by either a button and plunger type fixation component 52, as shown in FIG. 15, or by alternative fixation component 78, as shown in FIG. 19. Elastic tube 130 may also be attached to a rivet 31, as shown in FIGS. 7 and 8, or to a locking wire 39, as shown in FIG. 9, both described above. Dynamic components may also be attached between fixation components to create retention straps to stabilize tissue or structure to a base component or table.

System Utilization

Components of this invention can be used together to create surgical solutions, and may be used in a variety of combinations and mechanical and magnetic arrangements to achieve the requirements of a particular surgical procedure. Fixation components may adhere to base components, either by magnetic attraction or through use of weighted components, or to other structures such as operating tables, arm boards, laboratory tables, or clinical procedure work stations. The base component may comprise a base plate, either with or without a window, or may be a base component adapted for use in a particular procedure, such as smaller, coin-shaped plates that attach to skin or other surface using adhesive. Fixation components couple the dynamic, retraction and positioning components to base components. Retraction and positioning components engage tissue or other structure.

As shown in FIG. 13, a fixation component 52 may have a wire 70 and retractor 91 attached for static retraction, which is retraction that is constant for a given retractor position. Static retraction using fixation component 78 is illustrated in FIG. 18. Dynamic retraction may be achieved by applying an elastic tube 130 to a retractor and attaching the elastic tube to a fixation component, as shown in FIG. 15. Dynamic retraction using fixation component 78 is shown in FIG. 19. Such dynamic retraction can maintain a nearly constant force over a retractive range. For example, as a surgeon deepens an incision it is simultaneously further retracted. In short, unlike static retraction, it maintains a nearly constant force as an incision opens. This is ideal for incision retraction requiring repositioning or minor manipulation. In both cases, the fixation components may be moved as required.

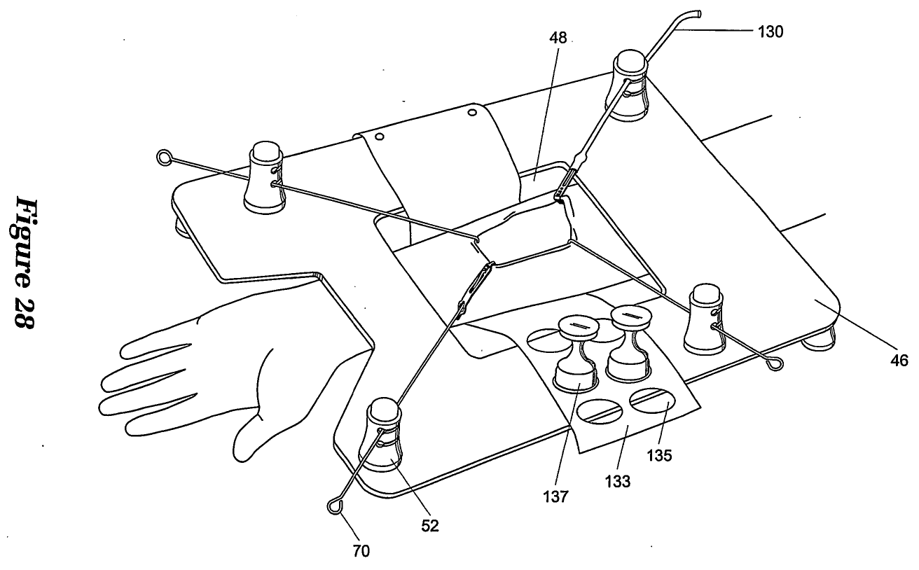

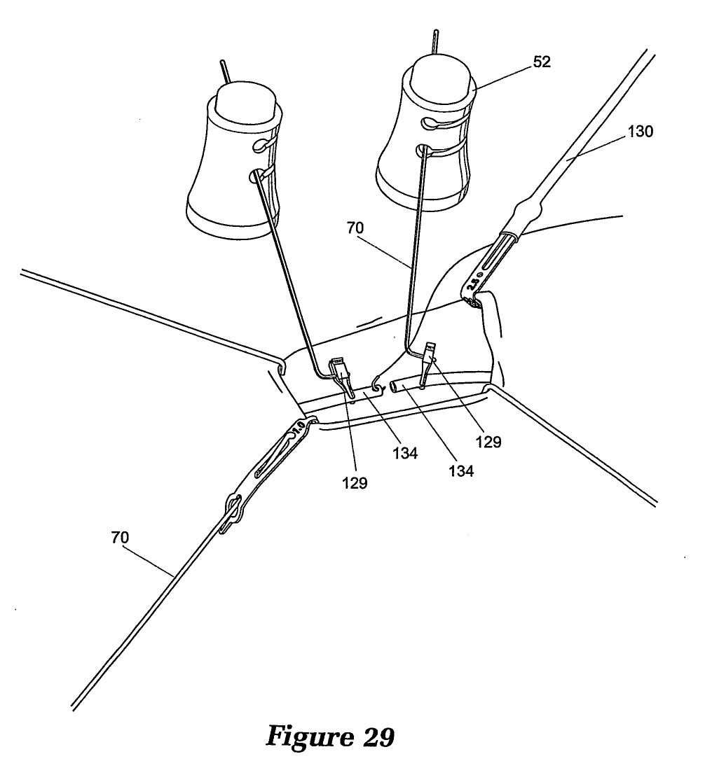

As shown in FIGS. 1, 28 and 29, components may also be combined to create a microsurgical system, providing retrieval and stabilization of structures such as small vessels or nerves. In a system for reapproximation of the severed ends of a vessel, the tissue may be stabilized using a belt 133 perforated at the ends. The belt 133 passes over and is secured to at least part of the base component 46, through the window 48 of the base and under a wrist, for example, so that the wrist is stable relative to the system. At least one end of the belt 133 should be adjustably attachable to the plate 46 using any suitable structure, such as holes 135, within which magnetic fixation components 137 are positioned. The tissue is the retracted using retraction and fixation components, as shown in FIG. 28. Finally, the surgeon manipulates the severed ends as shown in FIGS. 1 and 29. Wires 70 are coupled to magnetic fixation components 52 and engage microvascular clamps 129. Examples of microvascular clamps include: Bulldog™ clamps, S&T single use microvascular clamps™, Yasargil Aneurysm clips™, Fine Science Tools Micro-Serrefines™, or Beyer Micro Approximators™. A microvascular clamp 129 attached to a wire 70 engages the severed end of a vessel 134 in spring jaws, as shown in detail in FIG. 29. The wire 70 may be held or bent in place, and adjusted and held in position by one or all of the following: the wire may be bent or re-formed into a new shape; the wire may be shortened or lengthened in the fixator; the fixator may be moved on the surgical plate; the fixator may be rotated; the wire may be held in various slots at different heights; or adhesive plate base components 50 may be utilized in various positions or orientations to facilitate a variety of anchor locations and positions.

In the embodiment shown in FIG. 2 for use in a surgical procedure performed on a small animal, such as a mouse, a ferromagnetic base plate 47 having a window 48 is utilized together with other system components. A mouse 136 is placed at least partially within the window 48 and directly onto a body temperature maintenance system (not shown). Skin and other delicate tissue may be retracted by capturing one or more wires 70 in one or more magnetic fixation components 52. The prong of the retractor engages the tissue and the surgeon is able to stabilize the incision area by placing the magnetic fixation components 52 onto the ferromagnetic base plate 47. The attraction between the fixation component 52 and the base plate 47 is sufficient to allow stabilization of the surgical site, while permitting easy adjustment and manipulation of position of the fixation components 52. The force is also appropriate for the delicate small animal tissue. This system also provides secure positioning of probes or anesthesia inhalation components, so that the use of a stereotaxic frame may not be required.

In yet another embodiment for use during thoracic or any other specialty or general surgery, such as facial surgery, shown in FIG. 3, adhesive plate base components 50 are attached to the skin 138 using surgical adhesive. Magnetic fixation components 78 adhere to base components 50, which may be positioned on the patient, providing retraction in areas that are otherwise difficult to stabilize and manipulate. Fixation component 78 may secure a surgical drape 139 in position on the patient, as illustrated in FIG. 3. Structures may be created with these system components that allow the body of the patient to be used as an anchor point for retraction or approximation. In an alternative embodiment, fixation components 52 may be used with plates 50.

The systems of this invention are not confined to the embodiments described herein but includes variations and modifications within the scope and spirit of the foregoing description, the accompanying drawings, and the following claims. The components may be sized to accommodate a particular surgical procedure, so that larger or smaller versions of the components described herein may be used for various types of human and animal surgery. Additionally, numerous modifications in the shape and size of the described components can be made in order to adapt the principals of this invention to the anatomical and other requirements of surgical procedures on humans in addition to the procedures described and veterinary surgical procedures of all kinds.

1. A system comprising:
(a) at least one positioning component for engaging a tissue; and
(b) at least one fixation component for adjustable attachment to the positioning component.

2. The system of claim 1 wherein the positioning component comprises a wire.

3. The system of claim 2 wherein the wire comprises an end having a clamp.

4. The system of claim 3 wherein the clamp is adapted to atraumatically engage a vessel.

5. The system of claim 3 wherein the clamp is adapted to atraumatically engage a nerve.

6. The system of claim 1 wherein the fixation component comprises a magnet.

7. The system of claim 1 wherein the fixation component comprises:
(a) a housing comprising at least one slot;
(b) a plunger adapted to be received in the housing and comprising an opening; and
(c) a biasing element for holding the plunger in a first position;
wherein depressing the plunger moves the plunger from the first position to a second position wherein the opening of the plunger is aligned with the slot of the housing.

8. The system of claim 7 further comprising a magnet.

9. The system of claim 7 wherein the biasing element is a spring.

10. The system of claim 1 further comprising a base component

11. The system of claim 10 wherein the base component further comprises a window

12. The system of claim 10 wherein the fixation component is adjustably attached to the base component.

13. The system of claim 10 wherein the base component further comprises adhesive and wherein the base component is attached to the body.

14. A system comprising:
(a) a base component comprising a window;
(b) at least one fixation component for adjustable attachment to the base component; and
(c) at least one coupling component for adjustable attachment to the fixation component and for engaging the animal tissue.

15. The system of claim 14 wherein the fixation component further comprises:
(a) a housing comprising at least one slot;
(b) a plunger adapted to be received in the housing and comprising an opening; and
(c) a biasing element for holding the plunger in a first position;
wherein depressing the plunger moves the plunger from the first position to a second position wherein the opening of the plunger is aligned with the slot of the housing.

16. The system of claim 15 further comprising a magnet.

17. The system of claim 15 wherein the biasing element comprises a spring.

18. The system of claim 14 wherein the coupling component comprises a wire.

19. The system of claim 18 wherein the wire comprises an end having a hook.

20. A system comprising:
(a) at least one base component for attachment to tissue, the base component comprising adhesive;
(b) at least one fixation component for attachment to the base component; and
(c) at least one coupling component for attachment to the tissue and for adjustable attachment to the fixation component.

21. The system of claim 20 wherein the coupling component further comprises a retractor.

22. The system of claim 21 wherein the coupling component further comprises elastic tubing.

23. The system of claim 20 wherein the base component further comprises ferromagnetic material and the fixation component further comprises a magnet.

23. A fixation component comprising:
(a) a housing comprising at least one slot;
(b) a plunger adapted to be received in the housing and comprising at least one opening; and
(c) a biasing element for holding the plunger in a first position;
wherein depressing the plunger moves the plunger from the first position to a second position wherein the at least one opening of the plunger is aligned with the at least one slot of the housing.

24. The fixation component of claim 23 further comprising a magnet.

25. The fixation component of claim 23 wherein the biasing element comprises a spring.

26. A surgical retractor comprising a saddle-shaped tissue contacting surface perforated by holes and attached to an elongated member.

27. The retractor of claim 26 wherein the tissue contacting surfaces comprises metal.

28. The retractor of claim 26 wherein the tissue contacting surfaces comprises plastic.

29. A surgical kit for the manipulation and stabilization of tissue, comprising:
(a) at least one base component;
(b) at least one positioning component comprising a clamp; and
(c) at least one fixation component.

30. A surgical kit for the manipulation and stabilization of animal tissue; comprising:
(a) at least one ferromagnetic base component comprising a window;
(b) at least one fixation component for attachment to the base component, the fixation component comprising a magnet; and
(c) at least one coupling component for attachment to the tissue and for adjustable attachment to the fixation component.

31. A surgical kit for the manipulation and stabilization of tissue, comprising:
(a) at least one base component comprising an adhesive;
(b) at least one fixation component for attachment to the base component;
(c) at least one coupling component for attachment to the fixation component and for attachment to the tissue.

32. A method for manipulation or stabilization of tissue comprising:
(a) placing a base component proximate the tissue;
(b) coupling at least one fixation component to the base component;
(c) coupling a positioning component to the at least one fixation component; and
(d) coupling the positioning component to the tissue.

33. The method of claim 32 further comprising positioning a window of the base component over the tissue.

34. The method of claim 32 wherein the positioning component further comprises a clamp adapted to atraumatically engage the tissue.

35. A method of retracting or stabilizing animal tissue comprising:
(a) positioning at least part of an animal within a window of a base component;
(b) coupling at least one fixation component to the base component;
(c) coupling a retraction component to the at least one fixation component; and
(d) coupling the retraction component to the tissue.

36. The method of claim 35 wherein the retraction component further comprises a wire having an end comprising a hook.

37. The method of claim 35 wherein the retraction component further comprises a wire and a retractor, the retractor comprising a prong.

38. The method of claim 35 wherein the base component further comprises ferromagnetic material and the at least one fixation component further comprises a magnet.

39. A method of retracting or stabilizing tissue comprising:
(a) attaching a base component to a patient, the base component comprising adhesive;
(b) coupling at least one fixation component to the base component;
(c) coupling a retraction component to the at least one fixation component; and
(d) coupling the retraction component to the tissue.

40. The method of claim 39 wherein the base component further comprises ferromagnetic material and the at least one fixation component further comprises a magnet.

41. The method of claim 39 wherein coupling the retraction component to the fixation component is accomplished using elastic tubing.
